(19) United States
(12) Patent Application Publication
(10) Pub. No.: US 2004/0122413 A1
(43) Pub. Date: Jun. 24, 2004

(54) Absorbent article having shaped front and back ears

(75) Inventors: Thomas Harold Roessler, Appleton, WI (US); Georgia Lynn Zehner, Larsen, WI (US); Nancy Ellen Dawson, Appleton, WI (US); Emily Jean Baum, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/328,250

(22) Filed: Dec. 23, 2002

(51) Int. Cl. A61F 13/15; A61F 13/20

(52) U.S. Cl. 604/386

(57) ABSTRACT

The invention relates to absorbent articles (170) that have a pair of opposed back ear portions (190) extending laterally outward from the side edges of a back waist region of the article and a pair of opposed front ear portions (180) extending laterally outward from the side edges of a front waist region of the article. The back ear portions (190) and the front ear portions (180) are formed from the same material web (100). The material web (100) can include one or more types of materials. The back ear portions and the front ear portions have shape profiles that permit the back ear portions and the front ear portions to be nested with each other in the cross direction (215) and the machine direction (205) of the material web.

### BACKGROUND OF THE INVENTION

The present invention relates to disposable absorbent articles that include ear portions on the front and back waist regions where the ears are formed from the same web of material.

Billions of disposable diapers are sold and used every year in North America alone. Disposable diapers are one of the few consumer products that are made in such large quantities. Obviously, in order to efficiently and cost-effectively produce such a large number of disposable diapers, manufacturers of absorbent articles continually strive for process and design improvements.

Disposable diapers, and other absorbent articles, are typically manufactured from a high-speed moving web of nonwoven or other materials. Layers and components are introduced and brought together at various points to the moving web and, when all of the desired components are present, the web is cut into individual articles. As three-dimensional articles, absorbent articles are generally thin for comfort and fit purposes. Therefore, the primary dimensions of such articles are their length (longitudinal direction) and width (lateral direction). The thickness of an absorbent article is composed of piles of several of the article components that are generally coterminous with each other. The generally coterminous components include the outer cover (also known as the backsheet), the absorbent core and the bodyside liner (also known as the topsheet). The generally coterminous components of the absorbent article form the “chassis” of the absorbent article. Other components are often attached to the chassis to form the complete article. These separate components include containment flaps, leg elastics, waist elastics and fastening system elements. As can be generally appreciated, the complexity and difficulty of the absorbent article manufacturing process increases as the number of separate components that must be introduced to the moving web increases. An associated challenge is the placement of each of those components in the correct location on the high-speed moving web.

The chassis of an absorbent article is generally understood to include a front waist portion, a crotch portion and a back waist portion. Fastening systems have been developed for joining the waist portions together around the waist of the wearer of the article. In order to provide coverage of the article over the hips of the wearer and to improve placement of the fasteners, absorbent articles have evolved to include “ear” portions. The ear portions of a diaper can serve many functions; for example, the ear portions can aid placement of the waist portions prior to fastening and the ear portions can provide coverage of the article around the hips of the wearer of the article. With some absorbent articles, the ear portions can form at least a part of the waist opening and the leg openings.

The ear portions may be attached to the front waist portion, the back waist portion or there may be two sets-one in the front and one in the back. The ear portions generally extend laterally (perpendicularly) away from the chassis of the absorbent article. The ear portions can be made of a variety of materials and with disposable absorbent articles, they are typically made from a nonwoven material. At present, commercially available premium diapers tend to have back ear portions that are constructed from an elastic nonwoven material, such as a neck-bonded laminate. However, ear portions can be constructed of non-elastic or simply extensible nonwoven materials too and sometimes it is even desirable to have the ear portion constructed of a non-elastic nonwoven or for the ear portion to have a non-elastic/non-extensible portion attached to an elastic material. Therefore, in some cases, the ear portion can be constructed of more than one type of material. Including a non-elastic portion at the distal (furthest away from the chassis) end of the ear portion can aid the caregiver with application of the absorbent article because it gives them something sturdy to grab onto as they bring the waist portions of the article together for fastening.

While ear portions increase the ease with which absorbent articles can be applied and while they improve fit of the articles, there are limitations associated with their attachment to the chassis during manufacture. The limitations generally relate to the ear portions not being integral with the chassis and therefore, requiring separate placement onto the high speed moving web of materials. Typically, it is desirable to place the ear portions near the edges of the front and back waist portions. Consequently, it is necessary to “register” the ear portions to a desired location in relation to the front and back waist edges of individual absorbent articles. The closer the ear portions are to the waist edges, the better they perform their function. For example, if a diaper article includes a stretchable back waistband, registration of a stretchable back ear portion with the back waist edge results in better tensioning/performance of the stretchable waistband when the diaper is applied to a wearer. Unfortunately, current manufacturing limitations almost always prevent the ear portions from being exactly registered to the waist edges of the article. For example, current ear application systems often result in the ears and the fasteners attached to the ears being approximately 0.5 to 0.75 inches down from the waist edge of the diaper. The process of applying ear portions to the web is further complicated when the finished article is intended to include ears in both the front and the back waist regions. Commercially-available diapers having both front and back ear portions have front and back ears that are made from different types of nonwoven materials. Therefore, there are two, separate streams of ear portion materials that have to be introduced to the main web, further complicating manufacturing.

With many absorbent articles, it is desirable for the ear portions, whether placed in the front or back waist portion, to be curvilinear in shape. Because the ear portions extend perpendicularly away from the chassis, it may be desirable for the ear portions to have soft, curved edges in order to contribute to the articles overall “soft” appearance. If the shape of the ear is straight across or rectangular, the ear may bind into the upper thigh when a wearer is incertain positions such as a sitting position and the ear may cause red-marking and high-stress lines. Further, if the size of the ear is reduced by utilizing curved edges, the likelihood of exposing the wearers skin to mechanical fastening material that is part of the ear is reduced. For example, exposure of the wearers skin to a hook fastening material is less likely when the ear is smaller and when the fastener is landed closer to a side edge or a waist edge. Curved ears are desirable in the front waist region to resist the edges of the ears flipping over and to give a more uniform appearance. Even though the shape profile of ear portions on currently available diapers are curved, the options for shapes of ear portions are limited. Water cutters, which may be used to form ear portions, may not be able to provide a desirable, highly curved shape. Additionally, when forming highly curved ear portions using currently available methods, a lot of nonwoven material is wasted.

Currently available disposable absorbent articles are constructed from non-extensible chassis materials. However, the trend is to introduce more extensible and elastic components to absorbent articles in order to improve fit.

As ear portions become a standard feature of disposable absorbent articles, improvements to the current processes for applying ear portions to the moving web will be necessary. For example, there exists a need for improved registration of the ear portions with the front and back waist edges of the absorbent article. There also exists a need for greater flexibility in the shapes of ear portions that can be applied to the moving web of materials. Further, there exists a need for the front and back ear portions to be constructed from the same web of nonwoven material in order to simplify the absorbent article manufacturing process.

### SUMMARY OF THE INVENTION

In response to the difficulties and problems discussed above, new disposable absorbent articles that include front and back ear portions made from a single web of nonwoven source material have been invented. Further, the absorbent articles of the invention have front and back ear portions that are in improved registration with the front and back waist edges of the articles. When the front and back ear portions are registered with the waist edges of the article, the article achieves improved fit performance when worn. For example, if the article also includes a stretchable waistband, the article will fit better in the waist. Registration of the ear portions with the waist edges results in the waistband being better tensioned and consequently, results in the potential for improved leakage performance. Additionally, formation of front and back ear portions from the same material web permits the shapes of the front and back ear portions to be more highly curved and results in reduced trim waste of the nonwoven materials.

The present invention is directed in part to a disposable absorbent article that may define a front waist region, a back waist region and a crotch region that extends between and connects the front waist region and the back waist region. The disposable absorbent article may also define a longitudinal direction and a lateral direction. The absorbent article may include an absorbent chassis that defines an exterior surface and an interior surface opposite the exterior surface. The absorbent chassis may also define a pair of laterally opposed side edges and a pair of longitudinally opposed waist edges. Therefore, this configuration permits a waist edge to be associated with the front waist region of the article and a waist edge to be associated with the back waist region of the article. The absorbent article also may include a pair of opposed back ear portions formed from a material web, such as a nonwoven source material, and a pair of opposed front ear portions that are formed from the same material web as the opposed back ear portions. The pair of opposed back ear portions may extend laterally outward from the side edges of the absorbent chassis in the back waist region. The pair of opposed front ear portions may extend laterally outward from the side edges of the absorbent chassis in the front waist region.

The material web may include one or more nonwoven materials. For example, the material web may include a first material and a second material. The back ear portions may have a shape such that when they are formed from the material web, they include both the first material and the second material. Additionally, the front ear portions may have a shape such that when they are formed from the same material web, they include only the first material. Therefore, the shapes of the back ear portions and the front ear portions may be selected so that the shape of the back ear portion “nests” with the shape of the front ear portion. Selection of “nestable” shapes for the ear portions contributes to the feasibility of forming both the back ear portions and the front ear portions from the same material web. The material web may include a third material that is different from the first material or the second material. Any one of the materials within the material web may include a fastening material or have fastening capability and may be stretchable or extensible. The shapes of the back ear portions and the front ear portions may include curved edges so that the ear portions have curvilinear profiles.

The material web may include a machine direction and a cross direction. The back and front ear portions may have a length aligned with the cross direction of the material web and a width aligned with the machine direction of the material web. The shapes of the back and front ear portions may be selected such that the back ear portions have a length that is greater than the length of the front ear portions. The back and front ear portions may be attached to the side edges of the absorbent chassis using a known bonding technique such as adhesive, thermal or ultrasonic bonding. Further, the front ear portions on an article may be attached or bonded to the back ear portions on the article to form a prefastened absorbent article that is pulled up like underwear.

In another aspect, the present invention includes a disposable absorbent article having a back ear portion with a length and a front ear portion with a length that is less than the length of the back ear portion. The back ear portion and the front ear portion are formed from a material web and the back ear portion is opposite the front ear portion in a cross direction of the material web. The material web may include additional back ear portions and additional front ear portions. The back ear portions and the front ear portions may be adjacent to each other and alternate with each other in a machine direction of the material web. For example, the material web may be cut to form alternating shapes of back and front ear portions. Further, the material web may be cut and split to form two separate webs of alternating back and front ear portions. The material web may also be cut in such a way that a pattern of two front ear portions followed by two back ear portions is formed. The back ear portions and the front ear portions may have a width generally aligned with the machine direction of the material web. A mathematical sum of the width of a front ear portion and the width of an adjacent back ear portion (when the pattern that the material web is cut into is back ear: front ear: back ear: front ear, etc.) may be approximately equal to a sum of the width of an opposite back ear portion (opposite in the cross direction of the material web) and the width of an opposite front ear portion. The width of the front ear portion may be approximately equal to the width of the opposite front ear portion and the width of the adjacent (adjacent in the machine direction of the material web) back ear portion may be approximately equal to the width of an opposite back ear portion. The lengths of the back and front ear portions may be aligned with a cross direction of the material web. A mathematical sum of the length of a back ear portion and the length of an opposite and adjacent back ear portion maybe greater than a sum of the length of a front ear portion and the length of an opposite and adjacent front ear portion.

In another aspect, the present invention includes a unitary nonwoven web from which at least two disposable absorbent articles may be formed where the unitary nonwoven web includes a first longitudinal side and a second longitudinal side. The unitary nonwoven web includes a main composite of an absorbent structure and a barrier layer and a split material web. The split material web may include pairs of ear portions where each pair includes a back ear portion and a front ear portion. The pairs may be attached intermittently and opposite each other on both the first longitudinal side and the second longitudinal side of the unitary nonwoven web. The unitary nonwoven web may be separated into two or more disposable absorbent articles (such as diapers) at a severing point. The severing point may be located in the middle of the pairs, such as at a point between the back ear portion and the front ear portion of each pair. The nonwoven web may be separated in such a way that each of the disposable absorbent articles includes both two, opposed back ear portions and two, opposed front ear portions. If desirable, the two, opposed front ear portions of an individual article may be attached to the two, opposed back ear portions on the article to form a prefastened article that may be put on like pants. The severing point may result in edges of two adjacent articles such that the severing point forms a front waist edge of one article and a back waist edge of the other article.

In yet another aspect, the present invention includes a unitary nonwoven web from which two or more absorbent articles may be formed where the nonwoven web includes a first longitudinal side and a second longitudinal side. The nonwoven web includes a main composite of an absorbent structure and a barrier layer and a split material web. The split material web includes two or more pairs of back ear portions attached intermittently and opposite each other on both the first longitudinal side and the second longitudinal side of the nonwoven web and two or more pairs of front ear portions similarly attached. Each “pair” may include either two back ear portions or two front ear portions. The pairs alternate with each other. The unitary nonwoven web may be separated into two or more absorbent articles at a severing point located between two ear portions such that each article includes two, opposed back ear portions and two, opposed front ear portions. When the unitary nonwoven web is separated, two front waist edges are formed when the severing point is between a pair of front ear portions and two back waist edges are formed when the severing point is between a pair of back ear portions.

In another aspect, the present invention is directed to a method for making disposable absorbent articles. The absorbent articles may include a front waist region, a back waist region and a crotch region that extends between and connects the waist regions. The articles may also include a longitudinal direction and a lateral direction. The method of making may include a step of providing a first continuous web of nonwoven material(s). The first continuous web may be selectively cut to form two ear portion webs where each ear portion web includes a repeating pattern of a back ear portion and a front ear portion. The method may further include a step of crossing the two ear portion webs in such a way that they end up 180 degrees from their initial orientation. This step of crossining orients the back ear portions and front ear portions formed from the ear portion webs so that their shape profiles are facing laterally outward (outward in a cross direction of the ear portion web). The method may also include a step of selectively cutting the two ear portion webs to form pairs of ear portions where each pair includes a back ear portion and a front ear portion. Following the step of selectively cutting the ear portion webs, the method may include a step of providing a second continuous web of interconnected absorbent chassis. Each of the interconnected absorbent chassis may include an exterior surface, an interior surface opposite the exterior surface, a pair of laterally opposed side edges and a pair of longitudinally opposed waist edges. The method may also include a step of attaching pairs of ear portions on both of the laterally opposed side edges of the interconnected absorbent chassis. Therefore, a pair (one back ear portion and one front ear portion) is attached to both side edges. The pairs are attached intermittently so that the pairs of ear portions are aligned with the waist edges of the adjacent absorbent chassis. The method may further include a step of selectively cutting the second continuous web of interconnected absorbent chassis into discrete absorbent articles. Each discrete absorbent article may include two laterally opposed front ear portions and two laterally opposed back ear portions.

The ear portions formed by the method of the invention may each have a length. The length of the back ear portions may be greater than the length of the front ear portions. Further, the first continuous web that is part of the method may include a first nonwoven material and a second nonwoven material.

In another aspect, the present invention relates to a method of making disposable absorbent articles that are formed in a “front to front”/“back to back” configuration. With this method, the pairs of ear portions may include two front ear portions or two back ear portions. When the ear portions are attached, two front waist edges may be adjacent to each other and they may alternate with two back waist edges adjacent to each other. The method may include a step of providing a first continuous web and may include selectively cutting the first continuous web to form two ear portion webs. Each ear portion web may include a repeating pattern of two back ear portions and two front ear portions. The method may also include a step of crossing the two ear portion webs in such a way that they end up rotated 180 degrees from their initial orientation. The ear portion webs may be crossed so that the ear portions are flipped over to face laterally outward prior to attachment to the absorbent articles. The two ear portion webs may be selectively cut to form alternating sets of two back ear portions and two front ear portions. Therefore, each “set” includes either two back ear portions or two front ear portions. The method may also include a step of providing a second continuous web of interconnected absorbent chassis where each of the absorbent chassis includes an exterior surface, an interior surface opposite the exterior surface, a pair of laterally opposed side edges and a pair of longitudinally opposed waist edges. The method may also include a step of attaching the alternating sets of ear portions to both of the laterally opposed side edges of the interconnected absorbent chassis. For example, a set of two front ear portions may be attached to both side edges (one set opposite the other) at approximately the same positions on the side edges. The sets of ear portions may be attached so as to be in alignment with the waist edges of the adjacent absorbent chassis. The method may further include a step of selectively cutting the second continuous web of interconnected absorbent chassis into discrete absorbent articles. Each discrete absorbent article may include two laterally opposed front ear portions and two laterally opposed back ear portions.

It is to be understood that both the foregoing general description and the following detailed description are exemplary and are intended to provide further explanation of the invention claimed. The accompanying drawings, that are incorporated in and constitute part of this specification, are included to illustrate and provide a further understanding of the articles and methods of the invention. Together with the description, the drawings serve to explain various aspects of the invention.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively shows a method by which the articles of the invention may be made;

FIG. 2 representatively shows a pattern into which a composite material web may be cut in such a way that the back ear portions and front ear portions are nested together;

FIG. 3 representatively shows two individual absorbent articles adjacent each other as part of a continuous or unitary nonwoven web;

FIG. 4 representatively shows a composite material web from which nested front and back ear portions can be formed;

FIG. 5 representatively shows an alternative composite material web from which nested front and back ear portions can be formed;

FIG. 6 representatively shows another alternative composite material web from which nested front and back ear portions can be formed;

FIG. 7 representatively shows a plan view of a disposable absorbent article of the invention;

FIG. 8 representatively shows a further alternative composite material web from which nested front and back ear portions can be formed; and

FIG. 9 representatively shows a web of material from which front and back side ear portions can be formed for use on a prefastened absorbent article, such as a diaper pant.

### DETAILED DESCRIPTION OF THE INVENTION

The present invention relates to disposable absorbent articles that have attached front ear portions and back ear portions that are formed from the same material web. The material web can include one or more nonwoven or fastening materials. Typically, disposable absorbent articles include a front waist region, a back waist region and a crotch region that extends between and connects the waist regions. Absorbent articles also typically define a longitudinal direction and a lateral direction. Absorbent articles include an absorbent chassis that defines an exterior surface, an interior surface opposite the exterior surface, a pair of laterally opposed side edges and a pair of longitudinally opposed waist edges. As used herein, the term “disposable” refers to articles which are intended to be discarded after a limited use and that are not intended to be laundered or otherwise restored for reuse.

The present invention further relates to solving problems associated with the complexity of attaching front ear portions and back ear portions to the absorbent chassis of a disposable absorbent article, particularly when the front ear portions and the back ear portions are made of different materials. The articles of the invention have front ear portions and back ear portions that are in improved registration with the absorbent chassis. Further, articles of the invention also produce a reduced amount of waste materials because the front ear portions and the back ear portions are formed from a single web of material as opposed to two or more material webs.

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The disposable absorbent articles of the present invention will be described in terms of a disposable diaper article that is adapted to be worn by infants about the lower torso. In particular, the disposable absorbent articles will be described in terms of a disposable diaper having a pair of opposed back ear portions and a pair of opposed front ear portions. It is understood that the articles of the present invention are equally adaptable for other types of absorbent articles such as adult incontinent products, training pants, feminine hygiene products and other personal care or health care garments.

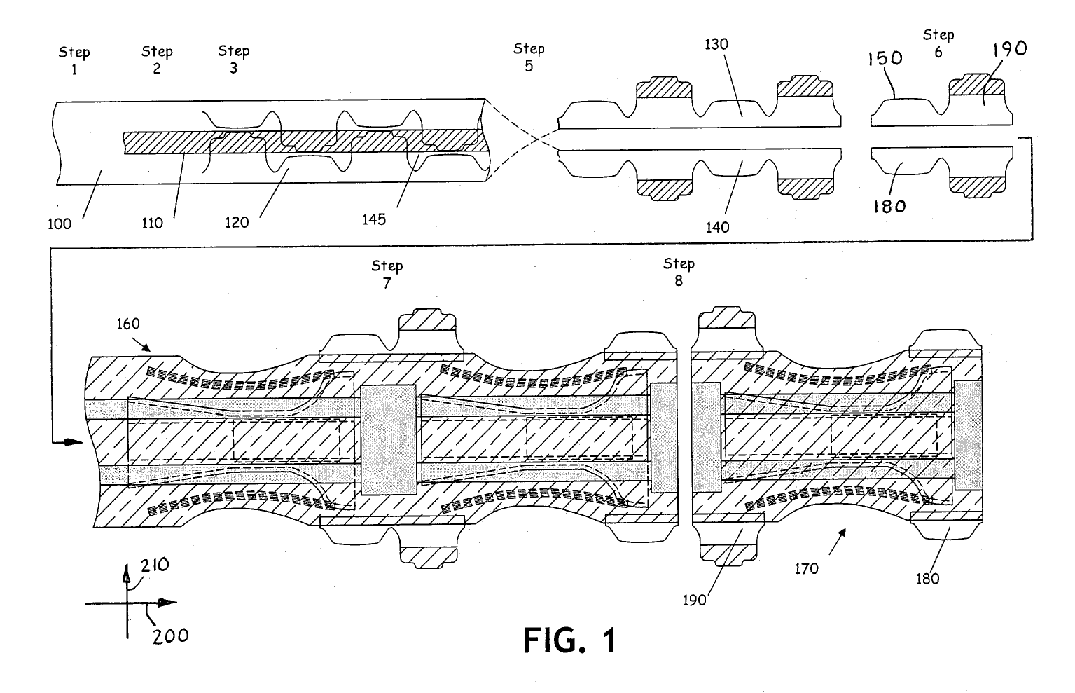

FIG. 1 representatively illustrates a method by which the disposable diapers of the invention can be made. Step 1 of the method includes providing a base web 100 of material to be used for forming the back ear portions and the front ear portions. An example of a suitable material for base web 100 is a 1.5 ounce per square yard (hereinafter “osy”) spunbond/meltblown/spunbond (hereinafter “SMS”) nonwoven material. Alternatively, the base web 100 may be formed of an extensible or an elastic material, such as a necked bonded laminate (hereinafter “NBL”). Further, the base web 100 may be formed of a combination of a generally non-extensible material and an extensible material such as a combination of an SMS and an NBL. Additional suitable materials for the base web 100 include “bonded carded webs” (“BCW”); heavy-weight spunbond; and film-based materials such as film-based or film-like based laminates. Typically, it is desirable to provide back ear portions or front ear portions with a fastening material so that one or the other of the ear portions is able to secure the back waist and front waist regions together. For such product configurations, a continuous web of a second material 110, that may be a fastening material, may be fed in association with and laminated to the base web 100 to form a composite material web 120. Lamination of a second material 110 to the base web 100 is illustrated as Step 2 in FIG. 1.

A next step for forming articles of the invention is to die cut the composite material web 120 into a right continuous composite web 130 and a left continuous composite web 140 in such a way that the back ear portions and the front ear portions being formed are nested with each other. Die cutting of the composite web 120 is illustrated in FIG. 1 as Step 3. The web material located between and not part of the back and front ear portions is referred to as trim waste 145. While not shown in FIG. 1, the trim waste is removed from the system. Next, shown as Step 5 in FIG. 1, right continuous composite web 130 and left continuous composite web 140 are transposed, that is the webs 130 and 140 are crossed so that the end up oriented 180 degrees opposite of their initial orientation. The right continuous composite web 130 and left continuous composite web 140 each have a sequence of back ear portions 190 and front ear portions 180. The sequence depicted in FIG. 1 of alternating back and front ear portions may be used with absorbent articles that are formed “back to front” (meaning the back or rear portion of one article is adjacent to the front portion of the next article). If the absorbent articles are being formed “front to front”/“back to back”, then the sequence of ear portions on the right and left continuous composite webs 130, 140 would be front, front, back, back etc. Step 6 of FIG. 1 depicts the right continuous composite web 130 and the left continuous composite web 140 being cut into discrete front and back ear segments (or pairs) 150. In Step 7, the front and back ear segments/pairs 150 are attached to a continuous absorbent article web 160. Finally, Step 8 of FIG. 1 depicts the continuous absorbent article web 160 being cut into individual absorbent articles 170. Each absorbent article 170 includes its own pair of opposed front ear portions 180 and pair of opposed back ear portions 190. A benefit of the front and back ear portions 180, 190 being made from the same composite web 120 is that the ear portions are applied to the article web 160 at the same time. Further, the front and back ear portions 180, 190 are in improved registration with the waist edges of the resulting absorbent articles 170.

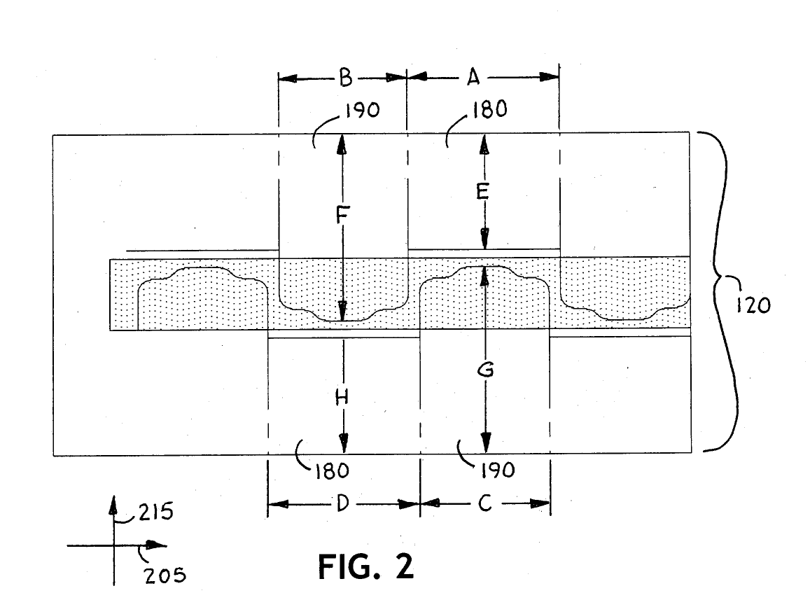

The absorbent article web 160 has a machine direction 200 and a cross-direction 210. FIG. 2 representatively depicts a pattern into which a composite web 120 may be cut such that the front and back ear portions 180, 190 are nested together. The ear portions have a length that is measured in direction 215 and a width that is measured in direction 205. When the ear portions are attached to the absorbent article web 160, the length of the ear portions is aligned with (parallel to) the cross-direction 210 of the absorbent article web 160 and the width of the ear portions is aligned with (parallel to) the machine direction 200 of the absorbent article web 160. Two of the back ear portions 190 depicted in FIG. 2 have lengths “F” and “G” and widths “B” and “C”. Two of the front ear portions 180 depicted in FIG. 2 have lengths “E” and “H” and widths “A” and “D”. As depicted in FIG. 2, the lengths “F” and “G” of the back ear portions 190 are greater than the lengths “E” and “H” of the front ear portions 180 that the back ear portions 190 are nested with. More specifically, the sum of lengths “F” and “G” is greater than the sum of lengths “E” and “H”. Additionally, the width “A” of one front ear portion 180 is approximately equal to the width “D” of the diagonally opposite front ear portion 180. Likewise, the width “B” of one back ear portion 190 is approximately equal to the width “C” of the diagonally opposite back ear portion 190. In another aspect of the relative dimensions of the front and back ear portions 180, 190, the sum of the widths “A” and “B” of adjacent back and front ear portions 180, 190 is approximately equal to the sum of the widths “C” and “D” of the opposing, adjacent back and front ear portions 180, 190.

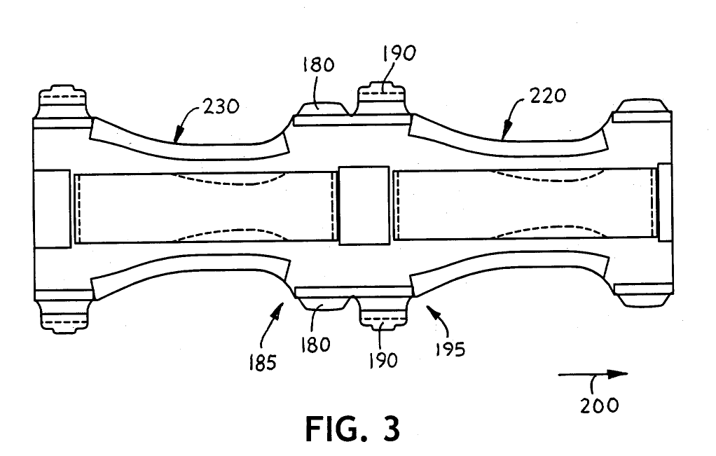

As previously described in relation to Step 8 of FIG. 1, the continuous absorbent article web 160 is cut into individual absorbent articles 170 at the “final cut off” point in the process. FIG. 3 depicts two adjacent articles from a representative continuous absorbent article web 160 immediately before the final cut off. At the final cut off, the front ear portions 180 will be separated from the back ear portions 190 that they were originally continuous with (based on formation from the composite web 120). The articles depicted in FIG. 3 are being formed in a “back to front” configuration; that is, the back waist region 195 of one article is adjacent the front waist region 185 of the next article. The machine direction 200 of the absorbent article web 160 is depicted in FIG. 3 in order to distinguish between the first article 220 formed and the second article 230 formed. Therefore, the back ear portions 190 attached to the first article 220 are adjacent the front ear portions 180 attached to the second article 230 and the adjacent ear portions are separated from each other when the first article 220 and the second article 230 are separated. When the first article 220 and the second article 230 are separated from each other at the final cut off, the cut off forms the waist or longitudinal edges of the adjacent articles. Use of the technique of the invention for applying ear portions results in the ear portions being in near perfect registration with the waist edges of the articles. That is, the attachment edge of the ear portion comes right to the waist edge instead of being somewhat over or below the waist edge. In a further processing step not depicted in FIG. 3, it is possible for front ear portions 180 of the articles to be attached to the back ear portions 190 of the articles to form prefastened disposable absorbent articles. The ear portions may be attached using techniques known to those of skill in the art including ultrasonic bonding, adhesives and mechanical fastening materials.

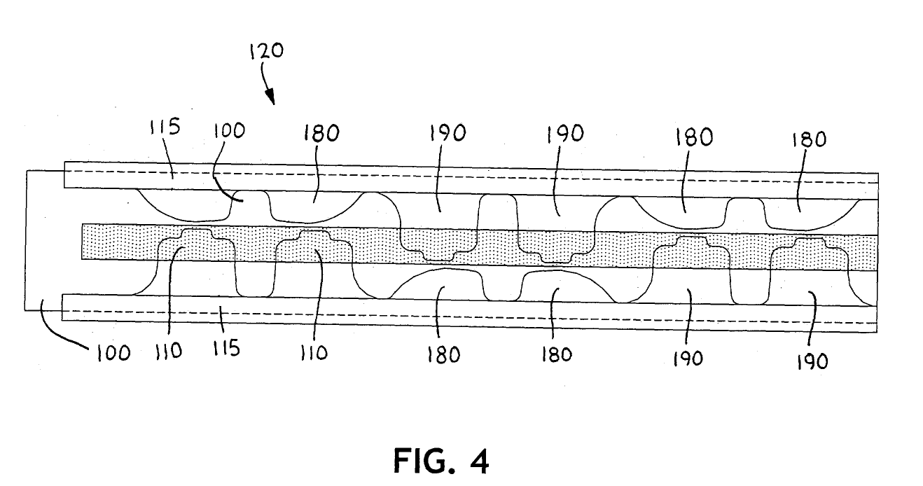

FIG. 4 representatively illustrates a composite web 120 from which nested front and back ear portions can be formed. The composite web 120 depicted in FIG. 4 representatively illustrates a nesting pattern for the front ear portions 180 and the back ear portions 190 of front ear portion 180; front ear portion 180; back ear portion 190; and back ear portion 190. When the composite web 120 is split into complementary right continuous composite web 130 and left continuous composite web 140, this nesting pattern may be suitable to form the ear portions of articles that are being formed in a front:front/back:back configuration. As shown, there are two front ear portions 180 on one side of the composite web 120 and there are two back ear portions 190 opposite the front ear portions 180. Once the composite web 120 is split for application to the absorbent article web 160, the right continuous composite web 130 may be sped up or slowed down in relation to the left continuous composite web 140 in order for sets of front ear portions 180 to be applied to adjacent front waist regions 185. Likewise, the relative speed of the right continuous composite web 130 and the left continuous composite web 140 should be timed so that sets of back ear portions 190 are applied to adjacent back waist regions 195.

In addition to depicting an alternative nesting pattern for the ear portions, FIG. 4 also depicts a composite web 120 that may include multiple materials for forming the ear portions. The composite web 120 includes a first material or base web 100. The base web 100 material may include a spunbond-meltblown-spunbond nonwoven material (“SMS” material). The composite web 120 may also include a second material 110 that is applied in such a way that it runs down the center of the composite web 120. The second material 110 may include a mechanical fastening hook material or other material suitable for mechanical fastening. By running the second material 110 down the center of the composite web 120, only the ends of the back ear portions 190 will include the second material 110. The length or height of the front ear portions 180 is selected so that it is short enough to not include the second material 110. The composite web 120 may further include a third material 115 that forms the middles of the back ear portions 190 and the ends of the front ear portions 180. Exemplary third materials may include a stretchable or extensible material or a super-soft material.

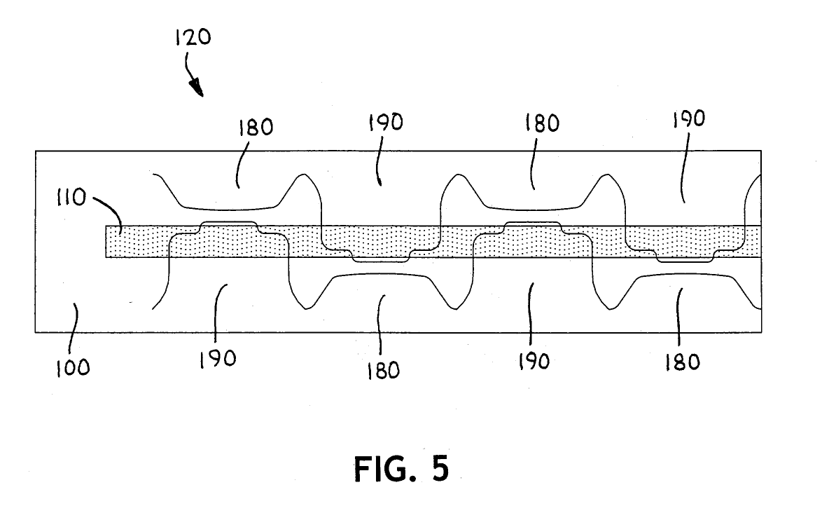

FIG. 5 representatively illustrates a composite web 120 that can be die-cut into front ear portions 180 and back ear portions 190. The shapes of the front ear portions 180 and the back ear portions 190 are selected so that the shapes can be “nested” with each other on the composite web 120. The nesting pattern depicted in FIG. 5 is similar to the pattern described in relation to FIG. 2. One difference is that the front ear portion 180 shape depicted in FIG. 5 includes a curved profile whereas the front ear portion 180 shape depicted in FIG. 2 includes a straight profile. The profiles of both the front ear portions 180 and the back ear portions 190 can be designed to have both curved and straight portions so long as the profiles can co-exist opposite each other when formed from the composite web 120. The nesting pattern of FIG. 5 is an example of how using the composite webs 120 of the invention can significantly reduce wasted materials; the front ear portions 180 and back ear portions 190 are closely nested together in such a way that there is minimal material remaining between them. The composite web 120 depicted in FIG. 5 includes both a base web 100 material and a second material 110. The second material 110 may be a fastening material that can be attached to or formed integrally with the base web 100. The fastening material can be formed of a soft, flexible polymer such as KRATON polymer, polyethylene, ethylene vinyl acetate, and metallocene film (adhesive-based) that can be printed or otherwise directly applied onto the base web 100.

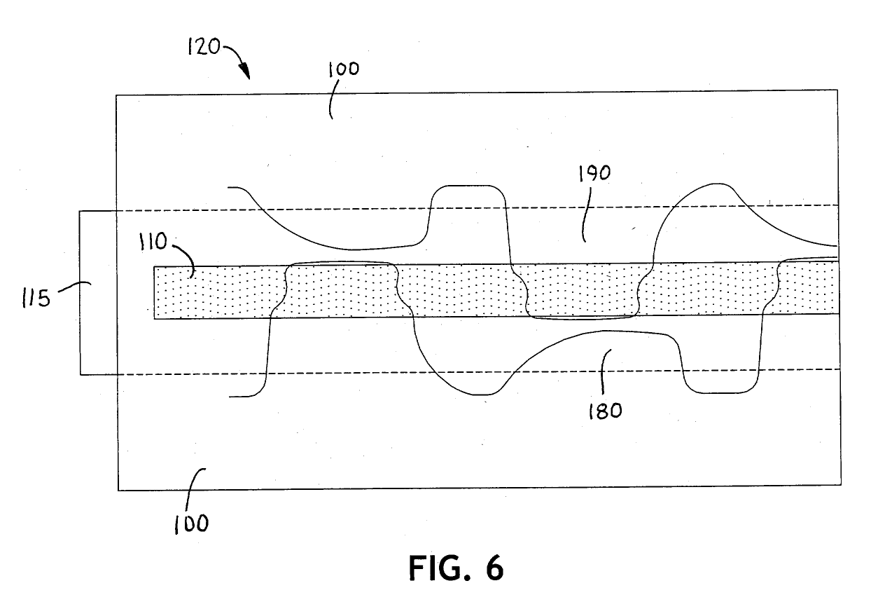

Another embodiment of front ear portions 180 and back ear portions 190 formed from the same material web is depicted in FIG. 6. FIG. 6 illustrates alternative shapes for both the front ear portions 180 and the back ear portions 190. These shapes illustrate that a variety of shapes may be selected and are still capable of being nested with each other on a common composite web 120. The use of multiple materials within one composite web 120 is also illustrated. For example, the composite web 120 may include a base web 100, a second material 110 and a third material 115. The base web 100 may form the parts of the front ear portions 180 and the back ear portions 190 that will become the attachment areas with an absorbent article web 160. A second material 110 may be attached to or formed integrally with the base web 100. The second materials may form the parts of the back ear portions 190 that will become a fastening surface for engagement with another fastening surface when used on an absorbent article. The second material 110 may be located on one side or surface of the base web 100 or on both sides/surfaces of the base web 100. A third material 115 may likewise be attached to or integrally formed with the base web 100. The third material 115 may be the same type of material as the second material 110 or it may be different. The third materials may form or run through a middle section of the back ear portions 190 and may form an end of the front ear portions 180. The third material 115 may be an extensible or elastic material such as a NBL or SBL. Both the second material 110 and the third material 115 may be located on one or more areas of the base web 100. In another aspect, the second material 110 may be attached to or formed integrally with the third material 115.

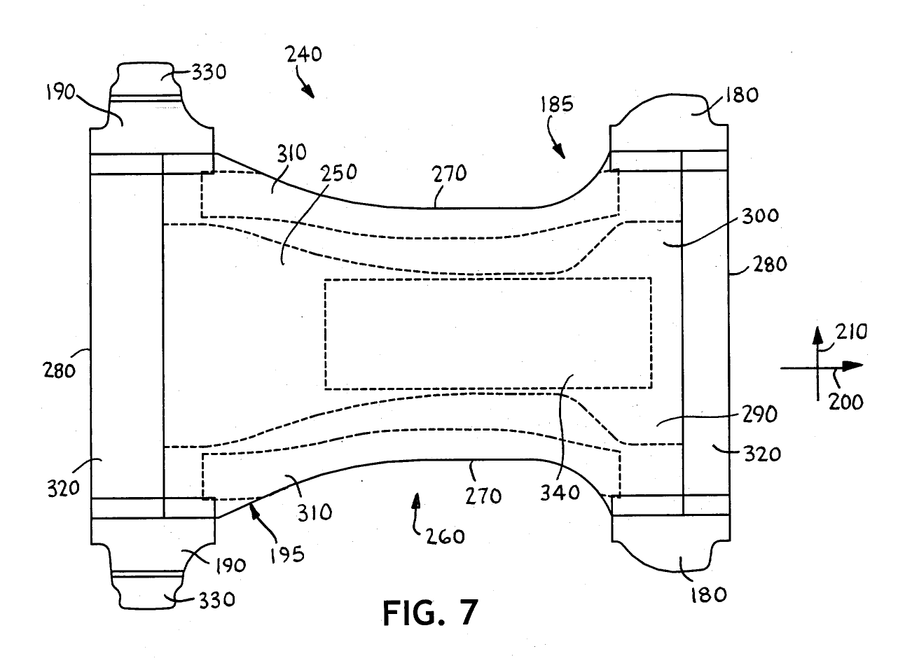

FIG. 7 representatively illustrates an example of a disposable absorbent article to which the front ear portions 180 and the back ear portions 190 formed from the same composite web 120 may be attached. The absorbent article, which may be in the form of a disposable diaper, is generally indicated at 240. As illustrated in FIG. 7, the diaper 240 defines an absorbent 250, a front waist region 185, a back waist region 195, a crotch region 260 that extends between and connects the front and back waist regions 185 and 195, a longitudinal direction 200 and a lateral direction 210. The front waist region 185 includes the portion of the diaper 240 that, when worn, is positioned on the front of the wearer while the back waist region 195 includes the portion of the diaper 240 that, when worn, is positioned on the back of the wearer. The crotch region 260 of the diaper 240 includes the portion of the diaper 240 that, when worn, is positioned between the legs of the wearer and covers the lower torso of the wearer.

The diaper 240 defines a pair of laterally opposed side edges 270, a pair of longitudinally opposed waist edges 280, an interior surface 290 that is configured to contact the wearer, and an exterior surface (not shown) opposite the interior surface 290 that is configured to contact the wearers clothing in use. The illustrated diaper 240 also includes an outer cover (not visible in FIG. 7) and a bodyside liner 300 that is connected to the outer cover in a superposed relation. An absorbent 250 is located between the outer cover and the bodyside liner 300. The laterally opposed side edges 270 of the diaper 240 are generally defined by the side edges of the outer cover that further define leg openings that may be curvilinear. The waist edges 280 of the diaper 240 are generally defined by the waist edges of the outer cover and define a waist opening that is configured to encircle the waist of the wearer when worn. The absorbent 250 is configured to contain and/or absorb any body exudates discharged from the wearer. The diaper 240 may further include leg elastics 310, containment flaps (not shown) and waist elastics 320 as are known to those skilled in the art. The diaper 240 may include fasteners 330 that are part of the back ear portions 190. It should be recognized that individual components of the diaper 240 may be optional depending upon the intended use of the diaper 240.

As illustrated in FIG. 7, the front and back ear portions 180 and 190 include curved edges and are in precise registration with the waist edges 280 of the diaper 240. More specifically, the front ear portions 180 are aligned with the waist edge 280 in the front waist region 185 of the diaper 240 and the back ear portions 190 are aligned with the waist edge 280 in the back waist region 195 of the diaper 240. This alignment or registration assists with improved fit and performance of the diaper 240. For example, when a diaper 240 such as the one illustrated in FIG. 7 is applied to a baby, the caregiver will pull (apply tension) the back ear portions 190 around to the front waist region 185 and secure the fasteners 330 to an exterior surface of the front waist region 185. Simultaneously, if the waist elastic 320 in the back waist region 195 is stretchable, the waist elastic 320 will be better tensioned and the diaper 240 will fit the baby better around the waist.

The diaper 240 may be of various suitable shapes. For example, in the unfastened configuration as illustrated in FIG. 7, the diaper may have an overall rectangular shape, T-shape or an approximately hourglass shape. In the shown embodiment, the diaper 240 has a generally I-shape in an unfastened configuration. Examples of diaper configurations suitable for use in connection with the instant application and other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,192,606 issued Mar. 9, 1993, to Proxmire et al., and U.S. Pat. No. 5,509,915 issued Apr. 23, 1996, to Hanson et al., the disclosures of which are herein incorporated by reference.

The various components of the diaper 20 are integrally assembled together employing various types of suitable attachment means, such as adhesive, sonic bonds, thermal bonds or the like, as well as combinations thereof. In the shown embodiment, for example, the outer cover and bodyside liner 300 are assembled to each other and to the absorbent 250 with adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Alternatively, the absorbent 250 may be connected to the outer cover using conventional fasteners such as buttons, hook and loop type fasteners, adhesive tape fasteners, and the like. The other components of the diaper 240 may be suitably connected together using similar means. Similarly, other diaper components, such as the elastic members 320, may be assembled into the diaper 240 article by employing the above-identified attachment mechanisms. Desirably, the majority of the diaper components are assembled together using ultrasonic bonding techniques for reduced manufacturing cost.

The outer cover of the diaper 240 may suitably be composed of a material which is either liquid permeable or liquid impermeable. It is generally preferred that the outer cover be formed from a material that is substantially impermeable to liquids. A typical outer cover can be manufactured from a thin plastic film or other flexible liquid-impermeable material. For example, the outer cover may be formed from a polyethylene film having a thickness of from about 0.013 millimeter (0.5 mil) to about 0.051 millimeter (2.0 mils). If it is desired to present the outer cover with a more clothlike feeling, the outer cover may be formed from a polyolefin film having a nonwoven web laminated to the exterior surface thereof, such as a spunbond web of polyolefin fibers. For example, a stretch-thinned polypropylene film having a thickness of about 0.015 millimeter (0.6 mil) may have thermally laminated thereto a spunbond web of polypropylene fibers. The polypropylene fibers have a thickness of about 1.5 to 2.5 denier per filament, which nonwoven web has a basis weight of about 17 grams per square meter (0.5 ounce per square yard). The outer cover may otherwise include bicomponent fibers such as polyethylene/polypropylene bicomponent fibers. Methods of forming such clothlike outer covers are known to those skilled in the art. The outer cover may also be an extensible outer cover such as the outer covers described in U.S. patent application Ser. No. 09/563,417 filed on May 3, 2000 by Roessler et al. The disclosure of application Ser. No. 09/563,417 is intended to be incorporated herein to the extent it is consistent with the present disclosure.

Further, the outer cover may be formed of a woven or nonwoven fibrous web layer which has been totally or partially constructed or treated to impart a desired level of liquid impermeability to selected regions that are adjacent or proximate the absorbent 250. Still further, the outer cover may optionally be composed of a micro-porous “breathable” material which permits vapors to escape from the absorbent 250 while still preventing liquid exudates from passing through the outer cover. For example, the outer cover may include a vapor permeable non-woven facing layer laminated to a micro-porous film. Suitable “breathable” outer cover materials are described in U.S. Pat. No. 5,695,868 issued to McCormack et al. and U.S. Pat. No. 5,843,056 issued Dec. 1, 1998 to Good et al., the descriptions of which are hereby incorporated by reference. Still further, the outer cover may also be an elastomeric material such as a stretch-thermal laminate (STL), neck-bonded laminate (NBL), or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman, and European Patent Application No. EP 0 217 032 published on Apr. 8, 1987 in the names of Taylor et al., the disclosures of which are hereby incorporated by reference. The outer cover can also be embossed or otherwise provided with a matte finish to provide a more aesthetically pleasing appearance.

The bodyside liner 300, as representatively illustrated in FIG. 7, suitably presents a bodyfacing surface that is compliant, soft feeling, and nonirritating to the wearer's skin. Further, the bodyside liner 300 may be less hydrophilic than the absorbent 250, to present a relatively dry surface to the wearer, and may be sufficiently porous to be liquid permeable, permitting liquid to readily penetrate through its thickness. A suitable bodyside liner 300 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The bodyside liner 300 is suitably employed to help isolate the wearer's skin from liquids held in the absorbent 250. The bodyside liner 300 can also be made from extensible materials as are described in U.S. patent application Ser. No. 09/563,417 filed on May 3, 2000 by Roessler et al.

Various woven and nonwoven fabrics can be used for the bodyside liner 300. For example, the bodyside liner may be composed of a meltblown or spunbond web of polyolefin fibers. The bodyside liner 300 may also be a bonded-carded web composed of natural and/or synthetic fibers. The bodyside liner 300 may be composed of a substantially hydrophobic material, and the hydrophobic material may optionally be treated with a surfactant or otherwise processed to impart a desired level of wettability and hydrophilicity. In a particular embodiment of the present invention, the bodyside liner 300 is made from a nonwoven, spunbond, polypropylene fabric composed of about 1.5-2.5 denier fibers formed into a web having a basis weight of about 20 grams per square meter and a density of about 0.13 grams per cubic centimeter. The fabric may be surface treated with about 0.3 weight percent of a surfactant commercially available from Hodgson Textile Chemicals, Inc. under the trade designation AHCOVEL Base N-62. The surfactant may be applied by any conventional means, such as spraying, printing, brush coating or similar techniques. The surfactant may be applied to the entire bodyside liner 300 or may be selectively applied to particular sections of the bodyside liner 300, such as the medial section along the longitudinal centerline of the diaper, to provide greater wettability of such sections. The bodyside liner 300 may further include a lotion or treatment applied thereto that is configured to be transferred to the wearer's skin. Suitable compositions for application to the bodyside liner 300 are described in U.S. Pat. No. 6,149,934 that issued to Krzysik et al. on Nov. 21, 2000.

The absorbent 250 of the diaper 240, as representatively illustrated in FIG. 7, may suitably be composed of a matrix of hydrophilic fibers, such as a web of cellulosic fluff, mixed with particles of a high-absorbency material commonly known as superabsorbent material. In a particular embodiment, the absorbent 250 includes a matrix of cellulosic fluff such as wood pulp fluff and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent 250 to better contain and absorb body exudates. The concentration of the superabsorbent particles may also vary through the thickness of the absorbent 250. Alternatively, the absorbent 250 may include a laminate of fibrous webs and superabsorbent material or other suitable means of maintaining a superabsorbent material in a localized area.

The absorbent 250 may have any of a number of shapes. For example, the absorbent may be rectangular, I-shaped, or T-shaped. It is generally preferred that the absorbent 250 be narrow in the crotch region 260 of the diaper 240. It has been found that the absorbent 250 of the present invention is particularly useful when the width dimension in the crotch region 260 is from about 2.5 to about 12.7 centimeters (1.0 to about 5.0 inches), desirably no more than about 7.6 centimeters (3.0 inches) and more desirably no more than about 5.1 centimeters (2.0 inches). The narrow crotch width dimension of the absorbent 250 allows the absorbent 250 to better fit between the legs of the wearer. The size and the absorbent capacity of the absorbent 250 should be compatible with the size of the intended wearer and the liquid loading imparted by the intended use of the absorbent article.

The high-absorbency material can be selected from natural, synthetic, and modified natural polymers and materials. The high-absorbency materials can be inorganic materials, such as silica gels, or organic compounds, such as crosslinked polymers. The term “crosslinked” refers to any means for effectively rendering normally water-soluble materials substantially water insoluble but swellable. Such means can include, for example, physical entanglement, crystalline domains, covalent bonds, ionic complexes and associations, hydrophilic associations such as hydrogen bonding, and hydrophobic associations or Van der Waals forces.

Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and similar compounds. Mixtures of natural and wholly or partially synthetic absorbent polymers can also be useful in the present invention. Such high-absorbency materials are well known to those skilled in the art and are widely commercially available. Examples of superabsorbent polymers suitable for use in the present invention are SANWET IM 3900 polymer available from Hoechst Celanese located in Portsmouth, Va. and DOW DRYTECH 2035LD polymer available from Dow Chemical Co. located in Midland, Mich.

The high absorbency material may be in any of a wide variety of geometric forms. As a general rule, it is preferred that the high absorbency material be in the form of discrete particles. However, the high absorbency material may also be in the form of fibers, flakes, rods, spheres, needles, or the like. As a general rule, the high absorbency material is present in the absorbent body in an amount of from about 5 to about 90 weight percent based on total weight of the absorbent 250.

Optionally, a substantially hydrophilic tissue wrapsheet may be employed to help maintain the integrity of the airlaid fibrous structure of the absorbent 250. The tissue wrapsheet is typically placed about the absorbent body over at least the two major facing surfaces thereof and composed of an absorbent cellulosic material, such as creped wadding or a high wet-strength tissue. In one aspect of the invention, the tissue wrapsheet can be configured to provide a wicking layer that helps to rapidly distribute liquid over the mass of absorbent fibers comprising the absorbent body. The wrapsheet material on one side of the absorbent fibrous mass may be bonded to the wrapsheet located on the opposite side of the fibrous mass to effectively entrap the absorbent 250. There may also be a surge layer 340 located between the absorbent body 250 and the bodyside liner 300 to facilitate the distribution of fluid during intake.

The disposable diaper 240 may include a pair of containment flaps (not shown in FIG. 7) that are configured to provide a barrier to the lateral flow of body exudates. The containment flaps may be located along the laterally opposed side edges 270 of the diaper adjacent the side edges of the absorbent 250. Each containment flap typically defines an unattached edge that is configured to maintain an upright, perpendicular configuration in at least the crotch region 260 of the diaper 240 to form a seal against the wearers body. The containment flaps may extend longitudinally along the entire length of the absorbent 250 or may only extend partially along the length of the absorbent 250. When the containment flaps are shorter in length than the absorbent 250, the containment flaps can be selectively positioned anywhere along the side edges 270 of diaper 240 in the crotch region 260. In a particular aspect of the invention, the containment flaps extend along the entire length of the absorbent 250 to better contain the body exudates.

Such containment flaps are generally well known to those skilled in the art. For example, suitable constructions and arrangements for containment flaps are described in U.S. Pat. No. 4,704,116 issued Nov. 3, 1987, to K. Enloe, the disclosure of which is hereby incorporated by reference.

The diaper 240 of the different aspects of the present invention may further include elastics at the waist edges 280 and side edges 270 of the diaper 240 to further prevent leakage of body exudates and support the absorbent 250. For example, the diaper 240 of the present invention may include a pair of leg elastic members 310 that are connected to the laterally opposed side edges 270 of the diaper 240 in the crotch region 260. The diaper 240 may also include a pair of waist elastic members 320 that is connected to the longitudinally opposed waist edges 280 of the diaper 240. The leg elastics 310 and waist elastics 320 are generally adapted to fit about the legs and waist of a wearer in use to maintain a positive, contacting relationship with the wearer to effectively reduce or eliminate the leakage of body exudates from the diaper 240.

Materials suitable for use as the leg elastics 310 and waist elastics 320 are well known to those skilled in the art. Exemplary of such materials are sheets or strands or ribbons of a polymeric, elastomeric material that are adhered to the outer cover in a stretched position, or that are attached to the outer cover while the outer cover is pleated, such that elastic constrictive forces are imparted to the outer cover. The leg elastics 310 may also include such materials as polyurethane, synthetic and natural rubber.

The diaper 240 of the different aspects of the present invention may further include a fit panel (not illustrated in FIG. 7) superimposed adjacent to the waist edge 280 in at least one of the waist sections 185 and 195, to provide a more comfortable, contouring fit about the wearer. For example, the diaper 240 may include a fit panel superimposed adjacent the waist edge 280 on either the interior or exterior surface of the diaper 240. Or there may be a fit panel located on both surfaces of the diaper 240 simultaneously. The diaper may include a fit panel disposed in both waist sections 185 and 195 and desirably the diaper includes a fit panel in at least the rear waist section 195. Desirably, the fit panel is extensible or elastomeric. For example, the diaper 240 includes an elastomeric fit panel on the interior surface 290 of the diaper 240 that is configured to elongate in the lateral direction 210 to provide an improved fit and appearance of the absorbent article about the wearer. This is accomplished by providing a mechanism for the waist region to expand, thereby increasing the waist perimeter dimension to assist in applying the diaper 240 on the wearer. Desirably the elastomeric or extensible fit panel allows the waist perimeter dimension to expand at least about 20 percent. The fit panel is further capable of initially providing a conforming fit about the wearer and maintaining such fit throughout the use of such article. The fit panel is also configured such that the absorbent 250 has the ability to expand, contract and receive body exudates without adversely affecting the positioning of the fit panel and the article about the waist of the wearer. Thus, with such a fit panel, movements of the wearer may move the absorbent but do not adversely affect the overall positioning and fit of the article on the wearer. Such improved fit can result in reduced leakage from the absorbent article and a more aesthetically pleasing appearance. When the fit panel is located on the interior surface 290 it may also extend beyond the side edges of the absorbent 250 of the diaper 240 and be generally coterminous with the waist edge 280 of the diaper 240 in the respective waist section 185 or 195. When located on the interior surface 290 of the diaper 240, the fit panel may define a free edge that extends longitudinally inward towards the crotch region 260 of the diaper 240. In a particular embodiment the free edge of the fit panel is configured to remain at least partially unattached to the bodyside liner 300 of the diaper 240 when in use to allow the absorbent 250 to move and expand to receive and contain body exudates. The unattached free edge may also form a pocket between the fit panel and the bodyside liner 300 that is configured to further contain body exudates. The free edge of the fit panel may be linear or curvilinear, such as concave, to better fit the wearer. The waist edge of the fit panel may also be curvilinear to better fit the wearer. Desirably, if the free edge is curvilinear, the waist edge 280 may also be curvilinear such that consecutive fit panels for multiple articles nest within each other and can be provided from a continuous sheet of material. In such a configuration, the free edge of the first fit panel corresponds to the waist edge of the next fit panel to improve manufacturing and reduce waste.

The fit panel may be provided in any suitable manner that provides the desired fit properties and performance. Desirably, the fit panel is an elastomeric or extensible material. The materials may include a SBL material, a NBL material, an elastomeric film, an elastomeric foam material or similar materials. The fit panel may be attached to the diaper 240 in any suitable manner that provides the desired properties. For example, the fit panel may be attached to the diaper using adhesive, ultrasonic, thermal bonding techniques and the like or combinations thereof. Absorbent articles including such a fit panel and methods of making the same are further described in PCT Patent Application WO 97/48357 published Dec. 24, 1997 and entitled “ABSORBENT ARTICLE HAVING FIT PANEL”, the disclosure of which is hereby incorporated by reference.

The diaper 240 of the different aspects of the present invention can further include a pair of fasteners 330 that may be used for joining the front and back waist sections 185 and 195 to each other during application of the diaper 240 to a wearer. An attachment panel (not illustrated in FIG. 7) may be located on the outer cover to which the fasteners 330 may be releasably engaged. The disposable diaper 240 of the present invention may include an attachment panel located on the outer cover in one of the waist regions 185 and 195 on the exterior surface of the diaper 240. In such a configuration, the fasteners 330 may be refastenably engaged with the attachment panel to maintain the diaper 240 about the waist of the wearer. The attachment panel may include two separate panels located along the opposed side edges of the diaper 240 in one of the waist regions 185 and 195 of the diaper 240. Alternatively, the attachment panel may include a single piece of material that extends substantially across the respective waist region of the diaper 240.

Suitable fastening materials to provide the engageable portions of the fasteners 330 are well known to those skilled in the art and can include adhesive tape tab fasteners, hook and loop fasteners, mushroom fasteners, snaps, pins, belts and the like, and combinations thereof. The fasteners 330 may include hook type fastening materials and the outer cover may be configured to function as a complementary loop type fastener. Alternatively, an attachment panel may be provided on the diaper 240 to function as a complementary loop type fastener. Desirably, the fasteners 330 include hook type fasteners that are releasably engageable directly with the outer cover of the diaper 240. Such an arrangement provides the ability to vary the size of the waist opening in very small increments over a wide range to fit the waist of the wearer. Particular examples of hook fastening materials can include VELCRO HTH 858 or VELCRO HTH 823, or a similar hook material available from Velcro Industries B.V., Amsterdam, Netherlands or affiliates thereof. The fasteners 330 may also include adhesive-type materials known in the art and having the desired strength for joining the waist regions 185 and 195 together when the diaper 240 is applied. Further, the fasteners 330 may include materials that are capable of cohesively attaching to or engaging an opposite portion of the diaper 240 for fastening about the wearer.

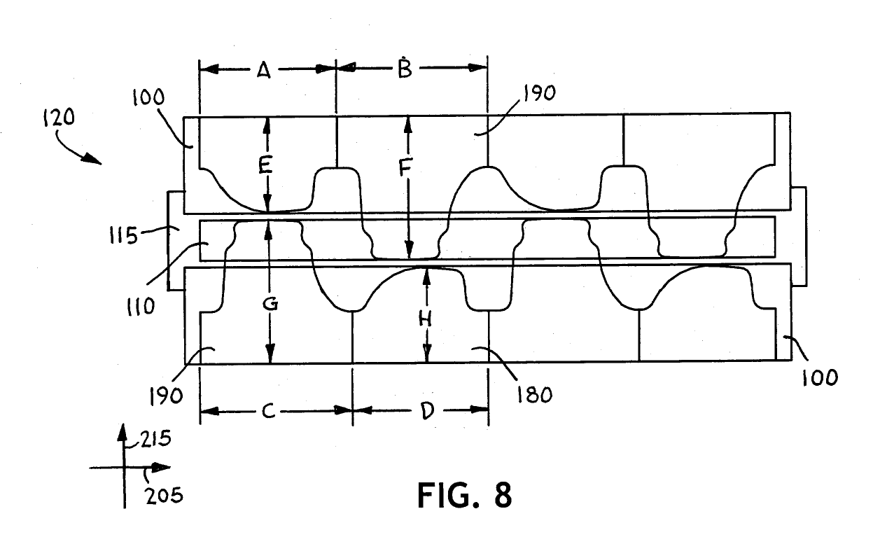

As previously described herein, the fastener 330 portion of the diaper 240 may be incorporated into the back ear portion 190 formed from a composite web 120 together with the front ear portion 180. FIG. 8 representatively illustrates an example of dimensions of front and back ear portions 180 and 190 that may be nested together and therefore formed from the same material web 120. The composite material web 120 depicted in FIG. 8 includes a base web 100 that may also be considered to be a “first” material, a second material 110 and a third material 115. The material web 120 has a machine direction 205 and a cross direction 215. The length of the front and back ear portions 180 and 190 is aligned with the cross direction 215 of the material web 120 and the width of the front and back ear portions 180 and 190 is aligned with the machine direction 205 of the material web 120. As representatively illustrated in FIG. 8, the third material 115 is underneath the base web 100 and the second material 110. The front ear portions 180 include the base web 100 and a portion of the third material 115. The front ear portions 180 do not, in this example, include the second material 110. The back ear portions 190 include the base web 100, the second material 110 and the third material 115. The base web 100 material is provided in two sections in this example; both sections are overlying the third material 115. Both sections of the base web 100 have a length in the cross direction 215 of the material web 120 of 55 mm. The second material 110 has a length in the cross direction 215 of the material web 120 of 23 mm. The third material 115 has a length in the cross direction 215 of the material web 120 of 56 mm.

Two diagonally opposed front ear portions 180 shown in FIG. 8 have widths represented by letters “A” and “D”. The widths of the front ear portions 180 are approximately 78 mm and “A” and “D” are approximately equal to each other. Two diagonally opposed back ear portions 190 shown in FIG. 8 have widths represented by letters “B” and “C”. The widths of the back ear portions 190 are approximately 86 mm and “B” and “C” are approximately equal to each other. The combined width of a pair of adjacent front and back ear portions 180 and 190 is represented by “A+B” and “C+D” is approximately equal to 164 mm. Further, the sum “A+B” is approximately equal to the sum “C+D”. The two diagonally opposed front ear portions 180 have lengths represented by letters “E” and “H”. The lengths of the front ear portions 180 are approximately 55 mm, approximately the same as the lengths of the base web 100 material in the cross direction 215. The two diagonally opposed back ear portions 190 have lengths represented by letters “F” and “G”. The lengths of the back ear portions 190 are approximately 78 mm, approximately the same as the sum of the lengths of one of the base webs 100 and the length of second material 110 plus the approximate gap between webs 100 and 110 in the cross direction 215. The lengths of the back ear portions 190 depicted in FIG. 8 also include a section of the third material 115. The lengths of the back ear portions 190 are generally greater than the lengths of the front ear portions 180; that is, 78 mm is greater than 55 mm. Additionally, the sum of the lengths of the back ear portions (“F+G”) is generally greater than the sum of the lengths of the front ear portions (“E+H”); that is, approximately 156 mm is greater than 110 mm. With the example of a nested ear pattern of the invention shown in FIG. 8, it may be desirable for the third material 115 to be a mechanical hook material that is capable of serving as a fastening material capable of engagement with another portion of an absorbent article to which the front and back ear portions 180 and 190 are eventually attached.

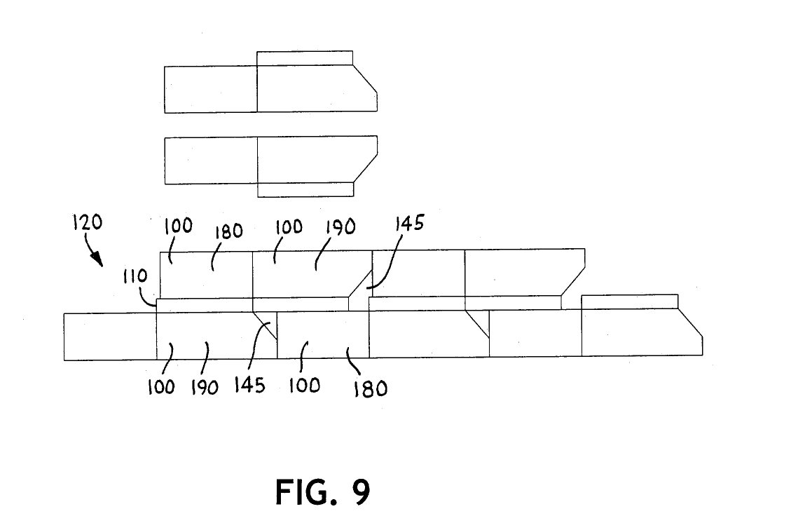

The present invention may also be utilized for the formation of prefastened absorbent articles. Prefastened articles may be pant-like in configuration so that the wearer puts their legs through leg openings and pulls the article up and over their hips. FIG. 9 representatively shows a material web 120 from which back ear portions 190 and front ear portions 180 may be formed for use on a prefastened absorbent article. The back ear portions 190 and front ear portions 180 may be more rectangular in shape and their nesting pattern may be such as to result in minimal trim waste 145. After the back ear portions 190 and front ear portions 180 are applied to an absorbent chassis, the back ear portions 190 may be attached to the front ear portions 180 in order to provide the “prefastened” configuration of the article. The back ear portions 190 and the front ear portions 180 may be attached by techniques know to those of skill in the art such as adhesive, thermal and ultrasonic bonding. Additionally, a second material 110 may be included in the composite material web 120 where the second material is a fastening material capable of engaging and attaching to the base web material 100 used to form the other portions of the front and back ear portions 180 and 190. For example, the second material 110 may be a mechanical hook material. As depicted in FIG. 9, the shapes of the front and back ear portions 180 and 190 may be selected such that only the back ear portions 190 include the second material 110.

While the invention has been described in detail with respect to specific aspects thereof, it will be appreciated that those skilled in the art, upon attaining an understanding of the foregoing, may readily conceive of alterations to, variations of and equivalents to these aspects. Accordingly, the scope of the present invention should be assessed as that of the appended claims and any equivalents thereto.

We claim:

1. A disposable absorbent article that defines a front waist region, a back waist region, a crotch region that extends between and connects the waist regions, a longitudinal direction and a lateral direction, the absorbent article comprising:
a) an absorbent chassis that defines an exterior surface, an interior surface opposite the exterior surface, a pair of laterally opposed side edges and a pair of longitudinally opposed waist edges;
b) a pair of opposed back ear portions that extend laterally outward from the side edges of the absorbent chassis in the back waist region, wherein the back ear portions are formed from a material web; and
c) a pair of opposed front ear portions that extend laterally outward from the side edges of the absorbent chassis in the front waist region, wherein the front ear portions are also formed from the material web.

2. The article of claim 1, wherein the material web comprises a first material and a second material.

3. The article of claim 2, wherein the back ear portions are formed from the first material and the second material and the front ear portions are formed from the first material.

4. The article of claim 2, wherein one of the first material and the second material is a fastening material.

5. The article of claim 1, wherein the material web has a machine direction and a cross direction and the back ear portions and the front ear portions have a length aligned with the cross direction of the material web and a width aligned with the machine direction of the material web.

6. The article of claim 5, wherein the length of the back ear portions is greater than the length of the front ear portions.

7. The article of claim 1, wherein the back ear portions have a curvilinear profile.

8. The article of claim 1, wherein the front ear portions have a curvilinear profile.

9. The article of claim 1, wherein the front ear portions in the front waist region are attached to the back ear portions in the back waist region to form a prefastened disposable absorbent article.

10. A disposable absorbent article having a back ear portion with a length and a front ear portion with a length less than the length of the back ear portion wherein the back ear portion and the front ear portion are formed from a material web, and further wherein the back ear portion is opposite the front ear portion in a cross direction of the material web.

11. The article of claim 10, wherein the material web further comprises additional back ear portions and additional front ear portions wherein the back ear portions and the front ear portions are adjacent and alternate with each other in a machine direction of the material web.

12. The article of claim 11, wherein each of the back ear portions and the front ear portions have a width aligned with the machine direction of the material web and further wherein a sum of the width of a front ear portion and the width of an adjacent back ear portion is approximately equal to a sum of the width of an opposite back ear portion and the width of an opposite front ear portion.

13. The article of claim 11, wherein the width of the front ear portion is approximately equal to the width of the opposite front ear portion; and the width of the adjacent back ear portion is approximately equal to the width of the opposite back ear portion.

14. The article of claim 11, wherein the lengths of the back ear portions and the front ear portions are aligned with the cross direction of the material web and further wherein a sum of the length of a back ear portion and the length of an opposite and adjacent back ear portion is greater than a sum of the length of a front ear portion and the length of an opposite and adjacent front ear portion.

15. A unitary nonwoven web from which at least two disposable absorbent articles may be formed including a first longitudinal side and a second longitudinal side comprising:
a main composite of an absorbent structure and a barrier layer; and
a split material web including pairs of a back ear portion and a front ear portion wherein the pairs are attached intermittently and opposite each other on both the first longitudinal side and the second longitudinal side of the unitary nonwoven web.

16. The unitary nonwoven web of claim 15 wherein the nonwoven web is separated into at least two disposable absorbent articles at a severing point between the pairs of back ear portions and front ear portions.

17. The unitary nonwoven web of claim 16 wherein each of the at least two disposable absorbent articles includes two, opposed back ear portions and two, opposed front ear portions.

18. The unitary nonwoven web of claim 17 wherein the two, opposed front ear portions of the articles are attached to the two, opposed back ear portions to form at least two prefastened disposable absorbent articles.

19. The unitary nonwoven web of claim 16 wherein the severing point forms a front waist edge of one disposable absorbent article and a back waist edge of the other disposable absorbent article.

20. A unitary nonwoven web from which at least two disposable absorbent articles may be formed including a first longitudinal side and a second longitudinal side comprising:
a main composite of an absorbent structure and a barrier layer; and
a split material web including at least two pairs of back ear portions attached intermittently and opposite each other on both the first longitudinal side and the second longitudinal side of the unitary nonwoven web and at least two pairs of front ear portions attached intermittently and opposite each other on both the first longitudinal side and the second longitudinal side of the unitary nonwoven web wherein the pairs of back ear portions and front ear portions alternate with each other.

21. The unitary nonwoven web of claim 20 wherein the nonwoven web is separated into at least two disposable absorbent articles at a severing point between the pairs of back ear portions and at a severing point between the pairs of front ear portions.

22. The unitary nonwoven web of claim 21 wherein each of the at least two disposable absorbent articles includes two, opposed back ear portions and two, opposed front ear portions.

23. The unitary nonwoven web of claim 21 wherein the severing point between the pairs of back ear portions forms back waist edges of the disposable absorbent articles and the severing point between the pairs of front ear portions forms front waist edges of the disposable absorbent articles.

24. The unitary nonwoven web of claim 22 wherein the two, opposed front ear portions of the articles are attached to the two, opposed back ear portions to form at least two prefastened disposable absorbent articles.

25. A method of making a disposable absorbent article that defines a front waist region, a back waist region, a crotch region that extends between and connects the waist regions, a longitudinal direction and a lateral direction, the method comprising:
a) providing a first continuous web;
b) selectively cutting the first continuous web to form two ear portion webs wherein each ear portion web includes a repeating pattern of a back ear portion and a front ear portion;
c) crossing the two ear portion webs;
d) selectively cutting the two ear portion webs to form pairs where each pair includes a back ear portion and a front ear portion;
e) providing a second continuous web of interconnected absorbent chassis wherein each of said absorbent chassis defines an exterior surface, an interior surface opposite the exterior surface, a pair of laterally opposed side edges and a pair of longitudinally opposed waist edges;
f) attaching the pairs of ear portions on both of the laterally opposed side edges of the interconnected absorbent chassis wherein the pairs of ear portions are attached in alignment with the waist edges of adjacent absorbent chassis; and
g) selectively cutting the second continuous web of interconnected absorbent chassis into discrete absorbent articles wherein each discrete absorbent article includes two laterally opposed front ear portions and two laterally opposed back ear portions.

26. The method of claim 25 wherein the front ear portions and the back ear portions each have a length and the length of the back ear portions is greater than the length of the front ear portions.

27. The method of claim 25 wherein the first continuous web includes a first nonwoven material and a second nonwoven material.

28. A method of making a disposable absorbent article that defines a front waist region, a back waist region, a crotch region that extends between and connects the waist regions, a longitudinal direction and a lateral direction, the method comprising:
a) providing a first continuous web;
b) selectively cutting the first continuous web to form two ear portion webs wherein each ear portion web includes a repeating pattern of two back ear portions and two front ear portions;
c) crossing the two ear portion webs;
d) selectively cutting the two ear portion webs to form alternating sets of two back ear portions and two front ear portions;
e) providing a second continuous web of interconnected absorbent chassis wherein each of said absorbent chassis defines an exterior surface, an interior surface opposite the exterior surface, a pair of laterally opposed side edges and a pair of longitudinally opposed waist edges;
f) attaching the alternating sets of ear portions to both of the laterally opposed side edges of the interconnected absorbent chassis wherein the alternating sets of ear portions are attached in alignment with the waist edges of adjacent absorbent chassis; and
g) selectively cutting the second continuous web of interconnected absorbent chassis into discrete absorbent articles wherein each discrete absorbent article includes two laterally opposed front ear portions and two laterally opposed back ear portions.

29. The method of claim 28 wherein the front ear portions and the back ear portions each have a length and the length of the back ear-portions is greater than the length of the front ear portions.

30. The method of claim 28 wherein the first continuous web includes a first nonwoven material and a second nonwoven material.
